(19) United States
(12) Patent Application Publication
(10) Pub. No.: US 2006/0263459 A1
(43) Pub. Date: Nov. 23, 2006

(54) Sea Buckthorn Compositions and Associated Methods

(75) Inventors: Jiashi Zhu, San Diego, CA (US); Yan Zhang, Beijing (CN)

Correspondence Address: THORPE NORTH & WESTERN, LLP., 8180 SOUTH 700 EAST, SUITE 200, SANDY, UT, 84070, US

(73) Assignee: Pharmanex, LLC, Provo, UT (US)

(21) Appl. No.: 11/382,902

(22) Filed: May 11, 2006

Related U.S. Application Data
(62) Division of application No. 10/821,262, filed on Apr. 7, 2004, now abandoned.
(60) Provisional application No. 60/462,354, filed on Apr. 10, 2003.

(51) Int. Cl. A61K 36/185

(52) U.S. Cl. 424/777

(57) ABSTRACT

Compositions having an effective amount of a Sea Buckthorn extract and an inert carrier and methods of use therefor are disclosed and described. Such methods of use include may include controlling serum lipid concentrations in a subject, and controlling the body weight of a subject, among others.

### PRIORITY DATA

This application is a divisional of U.S. patent application Ser. No. 10/821,262, filed on Apr. 7, 2004, which claims priority to U.S. Patent Application Ser. No. 60/462,354, filed on Apr. 10, 2003, which is incorporated herein by reference.

### FIELD OF THE INVENTION

The present invention relates to Sea Buckthorn extract compositions and uses thereof. Accordingly, the present invention involves the areas of botany, nutritional and health sciences, as well as medicine and pharmaceutical/nutraceutical sciences.

### BACKGROUND OF THE INVENTION

Physical fitness and good cardiovascular health have long been advocated by the medical community as factors that significantly enhance the quality and length of an individual's life. While ideal body weight will vary from person to person, general guidelines such as the Body Mass Index (BMI) have been established for determining whether a person's ideal weight. Many adverse health issues have now been directly linked to being overweight, especially with being obese.

The consequences of obesity include heighten risk of developing a number of adverse health conditions, such as type II diabetes, high blood pressure, heart disease, high cholesterol, breathing problems, sleep apnea, gallstones, arthritis, blood vessel problems, skin infections, rashes, sex hormone problems, gout, heartburn, gastroesophageal reflux disease (GERD), liver problems, and certain types of cancer. Obesity also often facilitates emotional problems, such as low self-esteem, depression, and certain eating disorders, and further prevents the obese individual from pursuing or engaging in various activities, such as swimming and other physical activities. As a result of the above-recited issues, obesity ultimately causes a significant decrease in overall quality of life, and increases the chance of premature death.

One of the adverse health conditions that is most directly linked to obesity is serum lipid disorders. While serum lipid disorders can be inherited, or otherwise experienced outside of obesity, the majority of serum lipid disorders are obesity induced. A variety of specific serum lipid disorders have been well established, such as elevated levels of triglycerides, cholesterol, and lipoproteins in the serum, commonly referred to as “high cholesterol,” “high triglycerides,” “hyperlipidemia,” and “acquired hyperlipoproteinemia.” One specific contributor to these lipid disorders can be excess ingestion of lipids or fatty substances. These lipid disorders have been linked to the development of atherosclerosis, heart disease, obesity, type I diabetes, type II diabetes, hypothyroidism, cushing's syndrome and renal failure. However, reduction of serum lipid concentrations to normal levels can reduce the health risks imposed by these conditions. Also, weight loss can be a factor in reducing these heath complications.

In view of the enormity of the obesity and weight problems faced by much of the world's population, a number of weight loss solutions have been sought. Myriads of special diets and exercise regimens have been developed. However, because of the considerable effort and perceived discomfort associated with dieting and exercise, a number of weight loss facilitating nutritional supplements and pharmaceutical formulations have been introduced.

Such dietary supplements and formulations have been touted to be fat trapping products, fat burning products, appetite suppressants, and laxatives or diuretics. Each of these individual categories of dietary supplements has unfavorable side effects, which decrease user compliance and most often do not result in the desired loss of weight. Fat trapping products, such as chitosan, supposedly work by preventing fat absorption into the body. Chitosan is derived from shellfish and may have allergenic potential with people with shellfish allergies. Fat burning products are claimed to increase the basal metabolic rate to burn calories, but this may not be safe for all people. MaHuang (Ephedra) are a class of these compounds and have been linked to high blood pressure, increased heart rate, heart palpitations, stroke, and even death. Also, laxative or diuretic abuse can be dangerous because it may result in excessive mineral loss and dehydration.

As a result, nutritional and pharmaceutical formulations that safely, and effectively, facilitate or contribute to weight loss continue to be sought through ongoing research and development efforts. Further, formulations that safely and effectively control or lower serum lipid concentrations continue to be sought

### SUMMARY OF THE INVENTION

Accordingly, the present invention provides formulations and methods for reducing body weight in a subject. In one aspect of the present invention, such a formulation may include, or consist essentially of, an effective amount of a Sea Buckthorn extract. In another aspect, additional active ingredients may be combined with the Sea Buckthorn extract. Such formulations may be suitably prepared into a number of dosage forms for administration to a subject by combination with yet additional ingredients such as an inert carrier. Examples of suitable dosage forms include without limitation oral, parenteral, and transdermal or transmucosal dosage forms.

In another aspect of the present invention, Sea Buckthorn formulations may be used in methods of controlling serum lipid concentrations in a subject. Such methods can include the steps of providing a composition containing a therapeutically effective amount of a Sea Buckthorn extract, and administering the composition to a subject. In some cases, controlling serum lipids may include lowering certain types of lipids in a subject, such as triglyceride, total cholesterol, and/or low-density lipoproteins. In other cases, controlling serum lipids may include elevating certain types of lipids, such as high-density lipoproteins

In another aspect of the present invention, Sea Buckthorn formulations may be used in methods of controlling the body weight of a subject. In some aspects, such methods can include providing a composition containing a therapeutically effective amount of sea buckthorn extract, and administering the composition to a subject. In some cases, controlling body weight may include reducing the body weight of the subject. In other cases, controlling body weight may include managing or maintaining a health weight by preventing weight gain.

In another aspect, the administration of Sea Buckthorn extract may stimulate production or release of cholecystokinin into the serum. Such activities may cause, or at least contribute to, the weight and serum lipid control effects recited herein.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the claims, or may be learned by the practice of the invention.

### DETAILED DESCRIPTION OF THE INVENTION

A. Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a carrier” includes reference to one or more of such carriers, and reference to “an excipient” includes reference to one or more of such excipients.

As used herein, the terms “formulation” and “composition” may be used interchangeably and refer to a combination of a pharmaceutically active agent with one or more additional ingredients such as an inert carrier. The terms “drug,” “active agent,” “bioactive agent,” “pharmaceutically active agent,” “nutraceutical active agent,” “pharmaceutical,” and “nutraceutical,” are also used interchangeably to refer to an agent or substance that has measurable specified or selected physiologic activity when administered to a subject in an effective amount. These terms of art are well known in the pharmaceutical, nutraceutical, and medicinal arts.

As used herein, “administration,” and “administering” refer to the manner in which a formulation or composition is introduced into the body of a subject. Administration can be accomplished by various art-known routes such as oral, parenteral, transdermal, inhalation, implantation, etc. Thus, an oral administration can be achieved by swallowing, chewing, sucking of an oral dosage form comprising the drug. Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. These and additional methods of administration are well known in the art.

The terms “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical, nutraceutical, herbaceutical, and health sciences. See, for example, Meiner and Tonascia, “Clinical Trials: Design, Conduct, and Analysis,” Monographs in Epidemiology and Biostatistics, Vol. 8 (1986), incorporated herein by reference.

The term “extract” when used in connection with a plant, refers to one or more active agents, or a composition containing such, that is obtained from the plant, or a portion thereof, including the flower, fruit, seed, peel, leaf, root, and bark. As will be recognized by those of ordinary skill in the art, extracts may be either crude or refined to a selected degree in order to isolate specified active agents. A number of extraction processes that can be employed to produce the compositions of various types will be recognized by those of ordinary skill in the art.

The term “Sea Buckthorn,” refers to the plant species hippophae rhamnoides, including all strains and hybrids thereof, grown anywhere in the world.

As used herein, “carrier” or “inert carrier” refers to a polymeric carrier, or other carrier vehicle with which a bioactive agent, such as a Sea Buckthorn extract, may be combined to achieve a specific dosage form. As a generally principle, carriers must not react with the bioactive agent in a manner which substantially degrades or otherwise adversely affects the bioactive agent. Other “inactive” ingredients may also be used in creating Sea Buckthorn extract formulations having specifically desired properties or dosage forms, and will be readily recognized by those of ordinary skill in the art.

As used herein, “subject” refers to a mammal that may benefit from the administration of a weight controlling and/or reducing, cholecystokinin serum concentration stimulating, or serum lipid controlling and/or reducing composition or method as recited herein. Most often, the subject will be a human.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a concentration range of 0.1 to 5 mg/kg should be interpreted to include not only the explicitly recited concentration limits of 0.1 mg/kg and 5 mg/kg, but also to include individual concentrations such as 0.2 mg/kg, 0.7 mg/kg, 1.0 mg/kg, 2.2 mg/kg, 3.6 mg/kg, 4.2 mg/kg, and sub-ranges such as 0.3-2.5 mg/kg, 1.8-3.2 mg/kg, 2.6-4.9 mg/kg, etc. This interpretation should apply regardless of the breadth of the range or the characteristic being described, and should apply to ranges having both upper and lower numerical values, as well as open-ended ranges reciting only one numerical value.

B. The Invention

Sea Buckthorn is known by the scientific name hippophae rhamnoides, and has been shown to be a source of many vitamins, including vitamin A, E, C, B1, B2, K, and P. It has also been reported that Sea Buckthorn is a significant source of various fatty acids, such as linoleic, alpha linoleic, oleic, palmitic, and palmitoleic acids. See, Yang et al., J. Nutr. Biochem. 10:622-630 (1999), which is incorporated herein by reference. These desirable ingredients such as antioxidants, carotenoids, carotenes, tocopherols, flavonoids, fatty acids, sterols and phytosterols, have attracted attention to Sea Buckthorn for a myriad of uses, such as in treatment of various skin conditions, prevention and treatment of cancer.

In accordance with the present invention, it has been discovered that extracts of Sea Buckthorn provide activity in controlling and/or reducing the body weight of a subject when administered in an effective amount. Further, it has been discovered that Sea Buckthorn extracts provide activity in controlling and/or lowering the serum lipid concentrations of a subject. Without wishing to be bound by theory, it is thought that the weight reducing activity may be attributed, at least in part, to the effect that a Sea Buckthorn extract has on stimulating production and/or release of cholecystokinin (CCK) in the subject, thereby increasing the serum concentration of cholecystokinin. As discussed in U.S. Pat. Nos. 6,207,638, and 6,429,190, each of which is incorporated herein by reference, cholecystokinin is a peptide that is released following the consumption of food, and is known to induce feelings of satiety and fullness. Cholecystokinin may also be involved in the rate of gastric emptying. Therefore, cholecystokinin stimulation may produce an appetite suppressing effect. Accordingly, the present invention provides compositions and methods for controlling and/or reducing the body weight of a subject, controlling and/or lowering the serum lipid concentrations of a subject, and stimulating release of cholecystokinin into the serum of a subject. Accordingly, an aspect of the present invention can include a Sea Buckthorn composition having a therapeutically effective amount of Sea Buckthorn extract and an inert carrier.

An aspect of the present invention includes methods of controlling serum lipid concentrations in a subject. Such methods may include providing a composition containing a therapeutically effective amount of a Sea Buckthorn extract and an inert carrier, and administering the composition to a subject. Accordingly, controlling the serum lipid concentration may be reducing serum lipid concentrations in a subject. In another aspect, controlling the serum lipid concentration may be preventing the serum lipid concentration from increasing. Additionally, the reduced serum lipids may be a triglyceride, total cholesterol, a low-density lipoprotein, and combinations thereof. In still another aspect, controlling the serum lipid concentration may be elevating high-density lipoprotein serum concentrations.

Another aspect of the present invention includes methods of controlling the body weight of a subject. Such methods may include providing a composition containing a therapeutically effective amount of Sea Buckthorn extract and an inert carrier, and administering the composition to a subject. In one aspect, controlling the body weight may be reducing the body weight of the subject. In another aspect, controlling the body weight may be preventing the body weight of the subject from increasing.

In an aspect of the present invention, the therapeutically effect amount of a Sea Buckthorn extract may increase the serum cholecystokinin concentration in the subject. In another aspect, the cholecystokinin serum concentration increase may occur by stimulating cholecystokinin production. In still another aspect, the cholecystokinin serum concentration increase may occur by an increased rate in the release of cholecystokinin from cholecystokinin producing cells. In a further aspect, the increased cholecystokinin serum concentration may cause appetite suppression.

In accordance with a Sea Buckthorn extract being a source for many nutritionally valuable vitamins, such as Vitamin C and Vitamin E, it has been discovered that compositions having a Sea Buckthorn extract can be used in a method of increasing immune function. For example, administration of a Sea Buckthorn extract may increase the function of lymphocytes. Accordingly, it is an aspect of the present invention to administer a therapeutically effective amount of a composition having a Sea Buckthorn extract and an inert carrier to a subject may increase the immune function of said subject.

Additionally, the presence of sterols within a Sea Buckthorn extract allows its use for cholesterol control. While not wishing to be bound to any particular theory, the mechanism of sterols on lowering cholesterol may be linked to the inhibition of cholesterol re-absorption from the gastrointestinal tract. Accordingly, phytosterols may inhibit the re-absorption of endogenous cholesterol, which may further lead to decreased serum levels of cholesterol. In an aspect of the present invention, the administration of a therapeutically effective amount of a composition having Sea Buckthorn extract to a subject may decrease the concentration of serum cholesterol.

Further, it is known that the age dependent degradation of the macular area of the retina, known as age-related macular degeneration or AMD, may be caused by light induced oxidation. Additionally, cataracts may be caused by light induced oxidation. As Sea Buckthorn extracts have been found be a source of antioxidants, including carotenoids, Vitamin C, Vitamin E, carotenoids, and others, one aspect of the present invention includes methods for providing compositions having Sea Buckthorn extracts as a source for antioxidants, which can be administered for eye health maintenance. In one aspect, a composition having a Sea Buckthorn extract can be administered as a source for antioxidants. In another aspect, a composition having Sea Buckthorn extract can be administered for ocular maintenance.

In accordance with the present invention, a composition that includes, or consists essentially of, a therapeutically effective amount of a Sea Buckthorn extract may be administered to a subject in order to obtain a desired weight controlling and/or reducing, serum lipid controlling and/or lowering, or cholecystokinin release stimulating effect. In one aspect, such a composition may consist essentially of a therapeutically effective amount of a Sea Buckthorn extract. In another aspect, the Sea Buckthorn extract may be combined with inert carriers and other inactive ingredients in order to create a specific dosage form. Accordingly, the inert carrier may be selected from the group consisting of calcium carbonate, calcium silicate, calcium magnesium silicate, calcium phosphate, kaolin, sodium hydrogen carbonate, sodium sulfate, barium carbonate, barium sulfate, magnesium sulfate, magnesium carbonate, activated carbon, water, isopropyl alcohol, ethyl alcohol, polyvinyl pyrrolidone, propylene glycol, polyethylene glycol stearyl alcohol, stearic acid, sorbitan monooleate, microcrystalline cellulose, sodium carboxymethyl cellulose, hydroxyethyl cellulose, hydroxypropyl cellulose, hydroxypropyl methylcellulose, sorbitol, mannitol, xylitol, starches, gelatins, lactose, acacia, carbomer, dextrin, guar gum, lactose, liquid glucose, maltodextrin, polymethacrylates, and combinations thereof.

In yet another aspect, such a composition may include additional active agents in addition to the Sea Buckthorn that are included to provide and intended effect, or specifically desired result. In still another aspect of the invention, the composition may further include an active ingredient selected from the group consisting of herbal extracts, botanical extracts, vitamins, minerals, amino acids, proteins, enzymes, and combinations thereof.

Examples of herbal extract agents and botanical extract agents that may be added to a Sea Buckthorn composition include without limitation, Ginseng, Ginko Biloba, Dong Quai, Hawthorn berry, St. John's Wort, Saw Palmetto, Kava Kava, Rose Hips, Echinacea, Licorice Root, Grape seed, Chammomile, Aloe Vera, Cinnamon Bark, Cordyceps, Ho Shou Wu, Dandelion, Gynostemma, mushroom, Notginseng, Dan Shen, etc. Additional examples of herbal extract can include, without limitation, Green tea plant, Causena Lansium, Crocus Sativus, Danshen (saliva miltiorrhize), Dongui (Radix angelicae sinesis), Eucommia, Evening primrose, Gastrodia elata, Hopes, Epimedium, Lemon balm, Mishmi bitter (coptis sinesis), Morning star (Uncaria rhychophylla), Passion flower, Physostigmine, Securinega Suffructicosa, Scutellaria baicalensis, Siberian cork tree (phellodendron amurense), Skullcap, Valerian, and mixtures thereof.

In an aspect of the present invention, fruit extracts and vegetable extracts can be included in a composition having a Sea Buckthorn extract. Examples of fruit extracts that may include apple, apricot, banana, blue berry, cranberry, cherry, fig, grape, grapefruits, hawthorn berry, huckleberry, kiwi fruit, kumquat, lemon, lime, mango, melon, nectarine, noni fruit, orange, papaya, peach, pear, persimmon, pineapple, plum, pomegranate, raspberry, strawberry, tangerine, watermelon, and mixtures thereof. Additionally, examples of vegetable extracts may include artichoke, avocado, asparagus, beans, bell pepper, broccoli, brussels sprout, cabbage, cauliflower, carrot, celery, cucumber, eggplant, green bean, lettuce, onion, parsley, pea, potato, pumpkin, radish, radicchio, rhubarb, spinach, tomato, zucchini, and mixtures thereof.

Some examples of acceptable vitamins that can be included in a composition with a Sea Buckthorn extract can include both water-soluble and oil soluble vitamins. Water-soluble vitamins can include B1, B2, B3, B4, B5, B6, B12, B13, B15, B17, biotin, choline, folic acid, inositol, para-amino benzoic acid (PABA), Vitamin C, Vitamin P, and mixtures thereof. Additionally, oil soluble vitamins include Vitamin A, Vitamin D, Vitamin E, Vitamin K, and mixtures thereof.

Also, examples of acceptable minerals that can be present in a composition having a Sea Buckthorn extract can include calcium, potassium, iron, chromium, phosphorous, magnesium, zinc, copper and mixtures thereof, as well as any other minerals essential to the human body.

Additionally, examples of acceptable amino acids include but are not limited to alanine arginine, carnitine, gamma-aminobutyric acid (GABA), glutamine, glycine, histidine, lysine, methionine, N-acetyl systeine, ornithine, phenylalanine, taurine, tyrosine, valine, and mixtures thereof.

Additionally, a composition containing a Sea Buckthorn extract can include additional antioxidants. Specific examples of acceptable antioxidants which can be incorporated into a Sea Buckthorn composition may include but are not limited to polyphenols such as catechin, beta-carotene, coenzyme Q10, grapnel, and mixtures thereof.

The Sea Buckthorn extract utilized in the present invention may be derived from any part of the Sea Buckthorn plant, and may take a variety of physical forms. However, in one aspect, the extract may be an oil. In another aspect, the extract may be a pulp. In yet another aspect, the extract may be water-soluble infusion extract. In a further aspect, the extract may be a dry, or lyophilized powder. In an additional aspect, the extract may be an emulsion. Moreover, the extract may be obtained from various portions of the Sea Buckthorn plant. In one aspect, the extract may be obtained from the fruit. In another aspect, the extract may be obtained from the leaves. In yet another aspect, the extract may be obtained from the stems or branches. In a further aspect, the extract may be obtained from the seeds. The Sea Buckthorn compositions of the present invention may be formulated into a variety of suitable dosage forms for the administration thereof. Many different dosage forms are well known to those of ordinary skill in the art and may be used for the administration of the Sea Buckthorn extract. In one aspect, the formulation may consist of the Sea Buckthorn extract prepared and administered to a subject directly. In another aspect, the extract may be combined with a suitable carrier and/or other inactive ingredients to provide a specific dosage form.

In one aspect, the Sea Buckthorn formulation may be provided as an oral dosage form. A variety of oral dosage forms are will known to those of ordinary skill in the art, and specific formulation ingredients may be selected in order to provide a specific result. Examples of oral dosage forms include without limitation, oral dosage forms, such as powders, tablets, capsules, gel capsules, liquids, syrups, elixirs, and suspensions. Additionally, oral dosage forms encompass food preparations, such as bars and beverages. Accordingly, in one aspect of the present invention, the Sea Buckthorn composition may be a dosage form selected from the group consisting of beverages, effervescent beverages, liquids, syrups, elixirs, suspensions, tablets, powders, capsules, gel capsules, confections, candies, bars, lozenges, and combinations thereof.

In another aspect, the Sea Buckthorn formulation may be provided as a transdermal or parenteral dosage form. A number of specific transdermal and parenteral dosage forms are known to those of ordinary skill in the art. Examples of transdermal dosage forms include without limitation, lotions, gels, creams, pastes, ointments, transmucosal tablets and adhesive devices, adhesive matrix-type transdermal patches, liquid reservoir transdermal patches, etc.

The amount of Sea Buckthorn extract included in the formulation need only be an amount that is sufficient to provide a desired therapeutic effect. As noted above, a variety of factors, such as individual physiology, the presence or absence of other compounds in the body, etc. may affect amount of Sea Buckthorn extract required to provide a therapeutic effect. Moreover, the amount of extract required to obtain weight reducing results may differ from the amount required to provide a serum lipid lowering effect. However, in one aspect, the amount of Sea Buckthorn extract may be an amount sufficient to stimulate the production and/or release of cholecystokinin. In another aspect, the amount may be sufficient to effect a body weight reduction. In another aspect, the amount may be sufficient to lower serum lipid concentrations. Those of ordinary skill in the art will be able to measure the physiological effect of the Sea Buckthorn extract and adjust the dosage amount accordingly.

As noted above, the Sea Buckthorn formulations of the present invention may optionally include one or more additional active agents. Both natural and synthetically produced active agents may be included. Those of ordinary skill in the art will be able to select from a wide range of specific ingredients in order to provide a desired therapeutic effect. In one aspect, the additional active ingredient may be a natural ingredient, such as an herbal or botanical extract. In another aspect, the additional active ingredient may be synthetically produced.

One specific type of additional active ingredient that may be used is an additional body weight reducing compound, such as a thermogenic compound (i.e. metabolism increasing compound). A wide range of compounds have been taught to produce a weight reducing effect, and are known to those of ordinary skill in the art. Examples of specific thermogenic compounds that may be used include without limitation, Ma Huang extract (ephedra), citrus aurantum extract (zhi shi, bitter orange, and synephrine), yohimbe extract (yohimbine), coleus extract (forskolin), and guarana (caffeine) and other stimulant compounds.

In another aspect of an embodiment of the invention, the additional active agent may be an essential dietary component, including without limitation vitamins and minerals, amino acids, proteins, and enzymes. Additional active ingredients that have a positive health imparting effect include without limitation, anti-inflammatory ingredients, natural analgesics, essential oils, antioxidants, and hormones. Further, anti-stress, or cortisol reducing agents, such as Ashwagandha, Beta-sitosterol, Epimedium, Garlic, L-Theanine, Magnolia bark extract, and Phosphatidylserine, as well as blood glucose modulating agents, such as corosolic acid may be included. A discussion of cortisol reducing compositions is included in copending patent application Ser. No. 60/390,424, which is incorporated by reference. A discussion of blood glucose modulating compositions is included in copending patent application Ser. No. 60/374,196, which is incorporated herein by reference.

As discussed above, the present invention additionally encompasses methods for using the Sea Buckthorn formulations disclosed herein. In one aspect, the present invention provides a method for stimulating cholecystokinin release in a subject, which includes administering an effective amount of a Sea Buckthorn extract to the subject. In another aspect, the present invention provides a method for reducing body weight in a subject, which includes administering an effective amount of a Sea Buckthorn extract to the subject. In yet another aspect, the present invention provides a method for lower serum lipids in a subject, which includes administering an effective amount of a Sea Buckthorn extract to the subject. Such extracts may be provided as part of any of the formulations disclosed herein, or may simply be administered directly to the subject.

In one aspect of the invention, the serum lipid lowering effect may lower total serum lipids. In another aspect, the serum lipids lowered may be triglycerides. In yet another aspect, the serum lipids lowered may be total cholesterol. In yet another aspect, the serum lipids lowered may be low-density lipids (LDL). As will be recognized by one of ordinary skill in the art, the extent of lowering, and actual lipids affected may be dictated by a number of factors, including Sea Buckthorn dosage amount, other active ingredients administered, level of initial serum lipid concentration, presence of interfering compounds in the serum, hereditary factors, etc.

While the above-recited formulations and methods have been primarily described in the context of treating a condition such as obesity or high serum lipids, it is to be understood that the present invention additionally encompasses methods for preventing such conditions. Such methods of prevention follow substantially the compositions and methods heretofore outlined, and may be further adjusted by one of ordinary skill in the art to more properly reflect a stratagem of maintenance or prevention, rather than of treatment. For example, the amount of Sea Buckthorn extract administered may be an amount sufficient to prevent or maintain the afore-mentioned conditions, rather than to abate them.

In accordance with the above described compositions and methods of use thereof, a Sea Buckthorn composition can be administered on a daily basis as needed or according to a specific and customized dosing regimen. Accordingly, administering the composition can include a single daily dose, and can further include multiple doses per day. In one aspect, administering the composition to the subject can be part of a sustained dosing regimen. In another aspect, the regimen can be less than about 1 year. In another aspect, the regimen can be less than about 6 months. In another aspect, the regimen can be less than about 3 months. In another aspect, the regimen can be less than about 1 month.

The examples provided below are illustrative of various embodiments of using Sea Buckthorn extract for weight loss and reduction of serum lipids in accordance with the present invention. While certain Sea Buckthorn extracts and/or additional ingredients, or combinations of ingredients, may be preferred, no limitation thereto is to be inferred. Rather, the type of Sea Buckthorn formulation desired will dictate which specific components, and amounts thereof, are included in addition to the Sea Buckthorn extract. It is to be understood the following examples were conducted on animals, where human formulations may vary with respect to the amount and concentration of any and/or all ingredient(s). These examples are provided to convey a more full understanding of the range of effective Sea Buckthorn formulations included in the present invention, and in no way to act as a limitation thereon.

### EXAMPLES

### Example 1

### High Calorie Rat Model

The effect of Sea Buckthorn on weight loss in a high calorie rat model was observed. Female Sprague-Dawley rats with an initial average weight of about 160 grams were fed with high calorie forage diet for 20 days. The high calorie forage consisted of lard (15%), sugar (10%), yolk powder (5%), and basic forage (70%). Each group consisted of 10 rats, and were administered with various formulations with or without Sea Buckthorn. The various formulations had the compositions are set forth in Table 1.

TABLE 1
Formulation Compositions

| Ingredient | A | B | | C | |
|---|---|---|---|---|---|
| Alisma Extract | 50 (mg/kg) | 50 | (mg/kg) | 50 | (mg/kg) |
| Epimedium Extract | 50 (mg/kg) | 50 | (mg/kg) | 50 | (mg/kg) |
| Semen Raphani Extract | | 1 | (g/kg) | 1 | (g/kg) |
| Sea Buckthorn Extract | | | | 2.5 | (ml/kg) |
| (fruit oil + seed oil, v/v = 1) | | | | | |

A comparison of the effects of Sea Buckthorn on the change in body weight of rats dosed with the various formulations is set for forth in Table 2.

TABLE 2
Impact of Sea Buckthorn on Body
Weight of High Calorie Fed Rats

| Dosage | | | |
|---|---|---|---|
| Day | A | B | C |
| 0 | 165.6 ± 10.38 (g) | 162.6 ± 7.6 (g) | 163.6 ± 11.27 (g) |
| 7 | 193.1 ± 8.45 | 196.3 ± 13.96 | 184.2 ± 11.06 |
| 14 | 228.8 ± 20.21 | 229.10 ± 15.52 | 202.7 ± 13.19 |
| 20 | 234.3 ± 17.22 | 232.6 ± 17.08 | 216.3 ± 14.53 |

A comparison of the rat group dosed with formulation A compared to the rate group dosed with formulation B showed there was not a significant change in the weight gain induced by the high calorie forage. However, when Sea Buckthorn was added, as in the rat group dosed with formulation C, it appeared to decrease the rate of weight gain induced by the high calorie forage in comparison with both the rat groups dosed with formulations A and B. Also, the addition of Sea Buckthorn showed a decrease in weight gain as early as the 7th day after initiation of feeding the rats the high calorie forage.

### Example 2

### Normal Mouse Model

The effect of Sea Buckthorn on weight loss in a normal mouse model was observed. Normal male Kun Ming mice with an initial average weight of about 18-22 grams were all fed with a normal calorie chow for 7 days. The mice groups were administered with various formulations with or without Sea Buckthorn. The dosages administered were: fruit juice (20 ml/kg), fruit oil (20 ml/kg, and seed oil (20 ml/kg). After 7 days the impact of fruit juice, fruit oil, or seed oil fractions of Sea Buckthorn on the body weight of the mice was observed compared to a control group not supplemented with Sea Buckthorn is set forth in Table 3.

TABLE 3
Impact of Sea Buckthorn on Body Weight of Normal Mice

| Dosage | Control | Fruit Juice | Fruit Oil | Seed Oil |
|---|---|---|---|---|
| Weight ± SD (g) | 27.4 ± 1 | 26.0 ± 3 | 25.1 ± 2 | 24.8 ± 3 |
| Number of mice | 9 | 9 | 8 | 9 |
| p (compared to control) | | 0.24 | 0.031 | 0.047 |

The results indicate that Sea Buckthorn can be effective in lowering the body weight of normal mice. More particularly, the results indicated that the Sea Buckthorn fruit oil and seed oil significantly lowered the body weight of mice fed with normal calorie chow.

### Example 3

### Sea Buckthorn Fruit Compound

The effect of a Sea Buckthorn fruit compound on weight loss in a normal mouse model was observed. Normal male Kun Ming mice with an initial average weight of about 18-22 grams were all fed with a normal calorie chow for 7 days. The mice groups were administered with or without a Sea Buckthorn fruit compound formulation. The Sea Buckthorn fruit compound formulation contained fruit powder (710 mg/kg), 10% crude flavone powder (80 mg/kg), and fruit oil (4 ml/kg). A comparison of the impact of a Sea Buckthorn fruit compound formulation on the body weight of the mice was observed compared to a control group not supplemented with Sea Buckthorn is set forth in Table 4.

TABLE 4
Impact of Sea Buckthorn on Body Weight of Normal Mice

| Dosage | Control | Sea Buckthorn Fruit Compound |
|---|---|---|
| Weight ± SD (g) | 29.5 ± 1 | 28.3 ± 1 |
| Number of mice | 12 | 12 |
| p (compared to control) | | 0.031 |

The results indicate that Sea Buckthorn can be effective in lowering the body weight of normal mice. More particularly, the results indicated that the Sea Buckthorn fruit compound significantly lowered the body weight of mice fed with normal calorie chow.

### Example 4

### Hyperlipidic Animal Models

The effect of Sea Buckthorn on the serum lipid concentrations in hyperlipidic animals was observed. Studies were conducted to determine the effect of Sea Buckthorn on serum lipids in various hyperlipidic animal models.

In one study, control and experimental rats were all fed a high-fat diet for 8 weeks, where the experimental group was administered with a formulated Sea Buckthorn product. The results of the study indicated that Sea Buckthorn decreased serum total cholesterol (TC) and decreased low-density lipoprotein cholesterol (LDL) by 30-36% in the experimental group compared to the control group (p<0.05).

In another study, acute hyperlipidemic mice were injected (i.p.) with 57% yolk emulsion (20 ml/kg). These mice were administered a formulated Sea Buckthorn product for seven days, which resulted in a 21-22% decrease in TC and LDL (p<0.05).

In another study, endogenous hyperlipidic rabbits were fed a cholesterol-free, casein-rich diet. These rabbits were also administered a formulated Sea Buckthorn product for 4 weeks, which did not result in significant changes in serum TC and LDL.

In another study, mixed endogenous-exogenous hyperlipidic rabbits showed Sea Buckthorn reduced serum TC and LDL by about 17-23% in comparison. The findings show Sea Buckthorn can be effective in substantially reducing both TC and LDL in exogenous hyperlipidemic animals.

Accordingly, it can be concluded that Sea Buckthorn extract was capable of reducing serum lipids. While not wishing to be bound by theory, it is believed that Sea Buckthorn may be capable of reducing serum TC and LDL by inhibiting or reducing the animal's capability of lipid absorption. Also, it is believed that Sea Buckthorn may be capable of reducing serum TC and LDL by accelerating the metabolism of cholesterols. Additionally, it is believed that Sea Buckthorn may be capable of reducing serum TC and LDL by accelerating the excretion of cholesterols. Further, it is believed that Sea Buckthorn may be able to reduce serum TC and LDL concentrations by any of these aforementioned processes alone or in combination.

It is to be understood that the above-described examples are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and/or preferred embodiments of the invention, it will be apparent to those of ordinary skill in the art that these examples not intended to be limiting in nature.

What is claimed is:

1. A method of controlling the body weight of a subject comprising:
(a) providing a composition containing a therapeutically effective amount of sea buckthorn extract and an inert carrier, and
(b) administering the composition to a subject.

2. A method as in claim 1, wherein the controlling is reducing the body weight of the subject.

3. A method as in claim 1, wherein the controlling is preventing the body weight of the subject from increasing.

4. A method as in claim 1, wherein the therapeutically effect amount of sea buckthorn extract increases serum cholecystokinin concentration in the subject.

5. A method as in claim 4, wherein the cholecystokinin serum concentration increase occurs by stimulating cholecystokinin production.

6. A method as in claim 4, wherein the cholecystokinin serum concentration increase occurs by an increased rate in the release from cholecystokinin producing cells.

7. A method as in claim 4, wherein the increased cholecystokinin serum concentration causes appetite suppression.

8. A method as in claim 1, wherein administering the composition to the subject is part of a sustained dosing regimen.

9. A method as in claim 8, wherein the regimen is less than about 1 year.

10. A method as in claim 9, wherein the regimen is less than about 6 months.

10. A method as in claim 9, wherein the regimen is less than about 3 months.

11. A method as in claim 10, wherein the regimen is less than about 1 month.

12. A method as in claim 1, wherein administering the composition to the subject includes a single daily dose.

13. A method as in claim 1, wherein administering the composition to the subject includes multiple doses per day.

14. A method as in claim 1, wherein the inert carrier is selected from the group consisting of calcium carbonate, calcium silicate, calcium magnesium silicate, calcium phosphate, kaolin, sodium hydrogen carbonate, sodium sulfate, barium carbonate, barium sulfate, magnesium sulfate, magnesium carbonate, activated carbon, water, isopropyl alcohol, ethyl alcohol, polyvinyl pyrrolidone, propylene glycol, polyethylene glycol stearyl alcohol, stearic acid, sorbitan monooleate, microcrystalline cellulose, sodium carboxymethyl cellulose, hydroxyethyl cellulose, hydroxypropyl cellulose, hydroxypropyl methylcellulose, sorbitol, mannitol, xylitol, starches, gelatins, lactose, acacia, carbomer, dextrin, guar gum, lactose, liquid glucose, maltodextrin, polymethacrylates, and combinations thereof.

15. A method as in claim 1, wherein the sea buckthorn extract is administered orally.

16. A method as in claim 15, wherein the oral administration is performed using a dosage form selected from the group consisting of beverages, effervescent beverages, liquids, syrups, elixirs, suspensions, tablets, powders, capsules, gel capsules, confections, candies, bars, lozenges, and combinations thereof.

17. A method as in claim 1, wherein the composition further comprises an active ingredient selected from the group consisting of herbal extracts, botanical extracts, vitamins, minerals, amino acids, proteins, enzymes, and combinations thereof.

18. A method as in claim 1, wherein the composition further comprises a cortisol controlling agent selected from the group consisting of ashwagandha, beta-sitosterol, Epimedium, garlic, L-theanine, magnolia bark extract, phosphatidylserine, and combinations thereof.
